SEQUENCE LISTING (13 sequences)

SEQ ID NO: 1 - protein, 706 aa, Homo sapiens
MASPADSCIQFTRHASDVLLNLNRLRSRDILTDVVIVVSREQFRAHKTVLMACSGLFYSIFTDQLKCNLSVINLDPEINPEGFCILLDFMYTSRLNLREGNIMAVMATAMYLQMEHVVDTCRKFIKASEAEMVSAIKPPREEFLNSRMLMPQDIMAYRGREVVENNLPLRSAPGCESRAFAPSLYSGLSTPPASYSMYSHLPVSSLLFSDEEFRDVRMPVANPFPKERALPCDSARPVPGEYSRPTLEVSPNVCHSNIYSPKETIPEEARSDMHYSVAEGLKPAAPSARNAPYFPCDKASKEEERPSSEDEIALHFEPPNAPLNRKGLVSPQSPQKSDCQPNSPTESCSSKNACILQASGSPPAKSPTDPKACNWKKYKFIVLNSLNQNAKPEGPEQAELGRLSPRAYTAPPACQPPMEPENLDLQSPTKLSASGEDSTIPQASRLNNIVNRSMTGSPRSSSESHSPLYMHPPKCTSCGSQSPQHAEMCLHTAGPTFPEEMGETQSEYSDSSCENGAFFCNECDCRFSEEASLKRHTLQTHSDKPYKCDRCQASFRYKGNLASHKTVHTGEKPYRCNICGAQFNRPANLKTHTRIHSGEKPYKCETCGARFVQVAHLRAHVLIHTGEKPYPCEICGTRFRHLQTLKSHLRIHTGEKPYHCEKCNLHFRHKSQLRLHLRQKHGAITNTKVQYRVSATDLPPELPKAC

SEQ ID NO: 2 - protein, 841 aa, Homo sapiens
MSVDEKPDSPMYVYESTVHCTNILLGLNDQRKKDILCDVTLIVERKEFRAHRAVLAACSEYFWQALVGQTKNDLVVSLPEEVTARGFGPLLQFAYTAKLLLSRENIREVIRCAEFLRMHNLEDSCFSFLQTQLLNSEDGLFVCRKDAACQRPHEDCENSAGEEEDEEEETMDSETAKMACPRDQMLPEPISFEAAAIPVAEKEEALLPEPDVPTDTKESSEKDALTQYPRYKKYQLACTKNVYNASSHSTSGFASTFREDNSSNSLKPGLARGQIKSEPPSEENEEESITLCLSGDEPDAKDRAGDVEMDRKQPSPAPTPTAPAGAACLERSRSVASPSCLRSLFSITKSVELSGLPSTSQQHFARSPACPFDKGITQGDLKTDYTPFTGNYGQPHVGQKEVSNFTMGSPLRGPGLEALCKQEGELDRRSVIFSSSACDQVSTSVHSYSGVSSLDKDLSEPVPKGLWVGAGQSLPSSQAYSHGGLMADHLPGRMRPNTSCPVPIKVCPRSPPLETRTRTSSSCSSYSYAEDGSGGSPCSLPLCEFSSSPCSQGARFLATEHQEPGLMGDGMYNQVRPQIKCEQSYGTNSSDESGSFSEADSESCPVQDRGQEVKLPFPVDQITDLPRNDFQMMIKMHKLTSEQLEFIHDVRRRSKNRIAAQRCRKRKLDCIQNLECEIRKLVCEKEKLLSERNQLKACMGELLDNFSCLSQEVCRDIQSPEQIQALHRYCPVLRPMDLPTASSINPAPLGAEQNIAASQCAVGENVPCCLEPGAAPPGPPWAPSNTSENCTSGRRLEGTDPGTFSERGPPLEPRSQTVTVDFCQEMTDKCTTDEQPRKDYT

SEQ ID NO: 3 - protein, 841 aa, synthetic construct
  REGION 1..841: note = mutant Bach2 sequence
MSVDEKPDSPMYVYESTVHCTNILLGLNDQRKKDILCDVTLIVERKEFRAHRAVLAACSEYFWQALVGQTKNDLVVSLPEEVTARGFGPLLQFAYTAKLLLSRENIREVIRCAEFLRMHNLEDSCFSFLQTQLLNSEDGLFVCRKDAACQRPHEDCENSAGEEEDEEEETMDSETAKMACPRDQMLPEPISFEAAAIPVAEKEEALLPEPDVPTDTKESSEKDALTQYPRYKKYQLACTKNVYNASSHSTSGFASTFREDNSSNSLKPGLARGQIKSEPPSEENEEESITLCLSGDEPDAKDRAGDVEMDRKQPSPAPTPTAPAGAACLERSRSVASPSCLRSLFSITKSVELSGLPSTSQQHFARSPACPFDKGITQGDLKTDYTPFTGNYGQPHVGQKEVSNFTMGSPLRGPGLEALCKQEGELDRRSVIFSSSACDQVSTSVHSYSGVSSLDKDLSEPVPKGLWVGAGQSLPSSQAYSHGGLMADHLPGRMRPNTSCPVPIKVCPRSPPLETRTRTSASCSSYSYAEDGSGGSPCSLPLCEFSSSPCSQGARFLATEHQEPGLMGDGMYNQVRPQIKCEQSYGTNSSDESGSFSEADSESCPVQDRGQEVKLPFPVDQITDLPRNDFQMMIKMHKLTSEQLEFIHDVRRRSKNRIAAQRCRKRKLDCIQNLECEIRKLVCEKEKLLSERNQLKACMGELLDNFSCLSQEVCRDIQSPEQIQALHRYCPVLRPMDLPTASSINPAPLGAEQNIAASQCAVGENVPCCLEPGAAPPGPPWAPSNTSENCTSGRRLEGTDPGTFSERGPPLEPRSQTVTVDFCQEMTDKCTTDEQPRKDYT

SEQ ID NO: 4 - protein, 825 aa, Homo sapiens
MLDICLEKRVGTTLAAPKCNSSTVRFQGLAEGTKGTMKMDMEDADMTLWTEAEFEEKCTYIVNDHPWDSGADGGTSVQAEASLPRNLLFKYATNSEEVIGVMSKEYIPKGTRFGPLIGEIYTNDTVPKNANRKYFWRIYSRGELHHFIDGFNEEKSNWMRYVNPAHSPREQNLAACQNGMNIYFYTIKPIPANQELLVWYCRDFAERLHYPYPGELTMMNLTQTQSSLKQPSTEKNELCPKNVPKREYSVKEILKLDSNPSKGKDLYRSNISPLTSEKDLDDFRRRGSPEMPFYPRVVYPIRAPLPEDFLKASLAYGIERPTYITRSPIPSSTTPSPSARSSPDQSLKSSSPHSSPGNTVSPVGPGSQEHRDSYAYLNASYGTEGLGSYPGYAPLPHLPPAFIPSYNAHYPKFLLPPYGMNCNGLSAVSSMNGINNFGLFPRLCPVYSNLLGGGSLPHPMLNPTSLPSSLPSDGARRLLQPEHPREVLVPAPHSAFSFTGAAASMKDKACSPTSGSPTAGTAATAEHVVQPKATSAAMAAPSSDEAMNLIKNKRNMTGYKTLPYPLKKQNGKIKYECNVCAKTFGQLSNLKVHLRVHSGERPFKCQTCNKGFTQLAHLQKHYLVHTGEKPHECQVCHKRFSSTSNLKTHLRLHSGEKPYQCKVCPAKFTQFVHLKLHKRLHTRERPHKCSQCHKNYIHLCSLKVHLKGNCAAAPAPGLPLEDLTRINEEIEKFDISDNADRLEDVEDDISVISVVEKEILAVVRKEKEETGLKVSLQRNMGNGLLSSGCSLYESSDLPLMKLPPSNPLPLVPVKVKQETVEPMDP

SEQ ID NO: 5 - protein, 654 aa, Homo sapiens
AEAPQVVEIDPDFEPLPRPRSCTWPLPRPEFSQSNSATSSPAPSGSAAANPDAAAGLPSASAAAVSADFMSNLSLLEESEDFPQAPGSVAAAVAAAAAAAATGGLCGDFQGPEAGCLHPAPPQPPPPGPLSQHPPVPPAAAGPLAGQPRKSSSSRRNAWGNLSYADLITKAIESSAEKRLTLSQIYEWMVKSVPYFKDKGDSNSSAGWKNSIRHNLSLHSKFIRVQNEGTGKSSWWMLNPEGGKSGKSPRRRAASMDNNSKFAKSRSRAAKKKASLQSGQEGAGDSPGSQFSKWPASPGSHSNDDFDNWSTFRPRTSSNASTISGRLSPIMTEQDDLGEGDVHSMVYPPSAAKMASTLPSLSEISNPENMENLLDNLNLLSSPTSLTVSTQSSPGTMMQQTPCYSFAPPNTSLNSPSPNYQKYTYGQSSMSPLPQMPIQTLQDNKSSYGGMSQYNCAPGLLKELLTSDSPPHNDIMTPVDPGVAQPNSRVLGQNVMMGPNSVMSTYGSQASHNKMMNPSSHTHPGHAQQTSAVNGRPLPHTVSTMPHTSGMNRLTQVKTPVQVPLPHPMQMSALGGYSSVSSCNGYGRMGLLHQEKLPSDLDGMFIERLDCDMESIIRNDLMDGDTLDFNFDNVLPNQSFPHSVKTTTHSWVSG

SEQ ID NO: 6 - protein, 684 aa, Homo sapiens
QLGEQLLVSSVNLPGAHFYPLESARGGSGGSAGHLPSAAPSPQKLDLDKASKKFSGSLSCEAVSGEPAAASAGAPAAMLSDTDAGDAFASAAAVAKPGPPDGRKGSPCGEEELPSAAAAAAAAAAAAAATARYSMDSLSSERYYLQSPGPQGSELAAPCSLFPYQAAAGAPHGPVYPAPNGARYPYGSMLPPGGFPAAVCPPGRAQFGPGAGAGSGAGGSSGGGGGPGTYQYSQGAPLYGPYPGAAAAGSCGGLGGLGVPGSGFRAHVYLCNRPLWLKFHRHQTEMIITKQGRRMFPFLSFNINGLNPTAHYNVFVEVVLADPNHWRFQGGKWVTCGKADNNMQGNKMYVHPESPNTGSHWMRQEISFGKLKLTNNKGANNNNTQMIVLQSLHKYQPRLHIVEVTEDGVEDLNEPSKTQTFTFSETQFIAVTAYQNTDITQLKIDHNPFAKGFRDNYDSSHQIVPGGRYGVQSFFPEPFVNTLPQARYYNGERTVPQTNGLLSPQQSEEVANPPQRWLVTPVQQPGTNKLDISSYESEYTSSTLLPYGIKSLPLQTSHALGYYPDPTFPAMAGWGGRGSYQRKMAAGLPWTSRTSPTVFSEDQLSKEKVKEEIGSSWIETPPSIKSLDSNDSGVYTSACKRRRLSPSNSSNENSPSIKCEDINAEEYSKDTSKGMGGYYAFYTT

SEQ ID NO: 7 - protein, 424 aa, Homo sapiens
MRIPVDPSTSRRFTPPSPAFPCGGGGGKMGENSGALSAQAAVGPGGRARPEVRSMVDVLADHAGELVRTDSPNFLCSVLPSHWRCNKTLPVAFKVVALGDVPDGTVVTVMAGNDENYSAELRNASAVMKNQVARFNDLRFVGRSGRGKSFTLTITVFTNPTQVATYHRAIKVTVDGPREPRRHRQKLEDQTKPFPDRFGDLERLRMRVTPSTPSPRGSLSTTSHFSSQPQTPIQGTSELNPFSDPRQFDRSFPTLPTLTESRFPDPRMHYPGAMSAAFPYSATPSGTSISSLSVAGMPATSRFHHTYLPPPYPGAPQNQSGPFQANPSPYHLYYGTSSGSYQFSMVAGSSSGGDRSPTRMLASCTSSAASVAAGNLMNPSLGGQSDGVEADGSHSNSPTALSTPGRMDEAVWRPYPAAKRVKLD

SEQ ID NO: 8 - protein, 187 aa, Homo sapiens
MPRVVPDQRSKFENEEFFRKLSRECEIKYTGFRDRPHEERQARFQNACRDGRSEIAFVATGTNLSLQFFPASWQGEQRQTPSREYVDLEREAGKVYLKAPMILNGVCVIWKGWIDLQRLDGMGCLEFDEERAQQEDALAQQAFEEARRRTREFEDRDRSHREEMEARRQQDPSPGSNLGGGDDLKLR

SEQ ID NO: 9 - protein, 20 aa, Foot-and-mouth disease virus
RAEGRGSLLTCGDVEENPGP

SEQ ID NO: 10 - protein, 20 aa, Foot-and-mouth disease virus
QCTNYALLKLAGDVESNPGP

SEQ ID NO: 11 - DNA, 10 bp, synthetic construct
  misc_feature 1..10: note = C/EBP transcription factor recognition sequence
  source 1..10: mol_type = other DNA
attgcgcaat

SEQ ID NO: 12 - DNA, 15 bp, synthetic construct
  misc_feature 1..15: note = NF-1 transcription factor recognition sequence
  variation 6..10: note = n is a, c, g, or t
  source 1..15: mol_type = other DNA
ttggcnnnnngccaa

SEQ ID NO: 13 - skipped

(19) United States
(12) Patent Application Publication
(10) Pub. No.: US 2024/0307440 A1
(43) Pub. Date: Sep. 19, 2024

(54) CELL EXPRESSING A CAR AND A TRANSCRIPTION FACTOR AND ITS USE

(71) Applicant: AUTOLUS LIMITED, London (GB)

(72) Inventors: Martin Pulé, London (GB); Simon Thomas, London (GB); Shaun Cordoba, London (GB); Wen Chean Lim, London (GB)

(21) Appl. No.: 18/525,271

(22) Filed: Nov. 30, 2023

Related U.S. Application Data
(63) Continuation of application No. 16/470,968, filed on Jun. 18, 2019, now abandoned.

(30) Foreign Application Priority Data
Dec. 21, 2016 (GB) 1621889.3

(51) Int. Cl. A61K 35/17; C07K 14/475; C07K 14/725; C12N 5/0783; C12N 15/85

(52) U.S. Cl. CPC A61K 35/17; C07K 14/475; C07K 14/7051; C12N 5/0636; C12N 15/85; C12N 2510/00

(57) ABSTRACT

The present invention provides a cell which comprises a first exogenous nucleic acid molecule encoding a Chimeric Antigen Receptor (CAR) and a second exogenous nucleic acid molecule encoding a transcription factor.

### CROSS-REFERENCE TO RELATED APPLICATIONS

This is a Continuation of U.S. application Ser. No. 16/470,968, filed Jun. 18, 2019, which is a U.S. National Phase of International Application No. PCT/GB2017/053834, filed Dec. 20, 2017, which claims priority to Great Britain Application No. 1621889.3, filed Dec. 21, 2016.

### INCORPORATION BY REFERENCE OF THE SEQUENCE LISTING

This application contains, as a separate part of disclosure, a Sequence Listing in computer-readable form (Filename: 52975A_SeqListing.xml; 18,305 bytes—XML file dated Dec. 8, 2023) which is incorporated by reference herein in its entirety.

### FIELD OF THE INVENTION

The present invention relates to a cell which co-expresses a chimeric antigen receptor (CAR) and a transcription factor. Expression of the transcription factor may prevent or reduce differentiation and/or exhaustion of the cell in vitro and/or in vivo.

### BACKGROUND TO THE INVENTION

Chimeric antigen receptors are proteins which graft the specificity of a monoclonal antibody (mAb) to the effector function of a T-cell. Their usual form is that of a type I transmembrane domain protein with an antigen recognizing amino terminus, a spacer, a transmembrane domain and an intracellular T-cell signalling domain. CAR-technology enables the generation of large numbers of T cells specific to any surface antigen. The cells are made by ex vivo viral vector transduction of, for example, a population of peripheral blood T-cells. Transduced cells, expressing the CAR may then be used for adoptive immunotherapy for the therapy of a disease.

It is necessary to activate T cells prior to transduction and to expand them in order to generate a clinically effective, therapeutic dose of T cells. Existing T cell activation/expansion methods are normally coupled with substantial T cell differentiation and can result in short-lived effects, including short-lived survival, lack of persistence and lack of in vivo expansion of the transferred T cells. To date, clinical efficacy of CAR-T cell immunotherapies is limited by poor T cell expansion and persistence after infusion into patients. There is therefore a need for improved therapeutic CAR-T cell compositions that survive, expand, and persist in vivo.

### DESCRIPTION OF THE FIGURES

FIG. 1—Schematic diagram illustrating the linear model of T-cell differentiation showing the expression markers associated with each cell type. APC—antigen-presenting cell; TCM—central memory T cell; TEFF—effector T cell; TEM—effector memory T cell; TN—naive T cell; TSCM—T memory stem cell.

FIG. 2—Graphs showing the proportion of Effector, Effector Memory (EM), Central Memory (CM) and Naïve cells following transduction (day 0) and 6 days after a 24 hour co-culture with CD19-expressing target cells (day 7). T cells were wither non-transduced (NT), transduced with a vector expressing the CAR only (HD37), or transduced with a vector expressing the CAR and the transcription factor FOXO1 (HD37-FOXO1). CD4+ and CD8+ subpopulations were analysed separately.

FIG. 3—Graphs showing the expression of CD27 and CD62L on CD4+ and CD8+ T cells 6 days after a 24 hour co-culture with CD19-expressing target cells. T cells were wither non-transduced (NT), transduced with a vector expressing the CAR only (HD37), transduced with a vector expressing the CAR and the transcription factor EOMES (HD37-EOMES) or transduced with a vector expressing the CAR and the transcription factor FOXO1 (HD37-FOXO1).

FIG. 4—Graphs showing the expression of CD62L on CD4+ and CD8+ T cells 6 days after a 24 hour co-culture with CD19-expressing target cells. T cells were wither non-transduced (NT), transduced with a vector expressing the CAR only (HD37), or transduced with a vector expressing the CAR and the transcription factors Runx3 and CBF beta (HD37-Runx3_CBFbeta).

FIG. 5—Graphs showing the proportion of Effector, Effector Memory (EM), Central Memory (CM) and Naïve cells following transduction (day 0) and 6 days after a 24 hour co-culture with CD19-expressing target cells (day 7). T cells were wither non-transduced (NT), transduced with a vector expressing the CAR only (HD37), transduced with a vector expressing the CAR and the transcription factor BACH2 (HD37-BACH2), or transduced with a vector expressing the CAR and a mutant version of the transcription factor BACH2 (HD37-BACH2_S520A). CD4+ and CD8+ subpopulations were analysed separately.

### SUMMARY OF ASPECTS OF THE INVENTION

In order for a CAR-T cell to be effective, it is important that it persists and expands in vivo and resists overly rapid differentiation and exhaustion. CAR T-cell persistence and engraftment is related to the proportion of naïve, central memory and T-stem-cell memory T-cells administered.

The present inventors have found that, by co-expressing the CAR with a transcription factor in a cell, it is possible to prevent or reduce differentiation and/or exhaustion of the cell. This results in a greater proportion of naïve, central memory and stem-cell memory cells in the cell composition for immunotherapy, and more effective persistence and expansion of the cells in vivo.

Thus, in a first aspect, the present invention provides a cell which comprises a first exogenous nucleic acid molecule encoding a Chimeric Antigen Receptor (CAR) and a second exogenous nucleic acid molecule encoding a transcription factor.

The transcription factor may prevent or reduce differentiation and/or exhaustion of the cell.

The transcription factor may be an effector memory repressor, such as BLIMP-1 Alternatively, the transcription factor may be a central memory repressor such as BCL6 or Bach2.

The transcription factor may be or comprise Bach2 or a modified version of Bach2 which has reduced or removed capacity to be phosphorylated by ALK. For example, modified Bach2 may comprise a mutation at one or more of the following positions with reference to the amino acid sequence shown as SEQ ID No. 2: Ser-535, Ser-509, Ser-520.

The transcription factor may be FOXO1.

The transcription factor may be EOMES.

The transcription factor may comprise Runx3 and/or CBF beta.

In a second aspect, the present invention provides a nucleic acid construct which comprises a first nucleic acid sequence encoding a Chimeric Antigen Receptor (CAR) and a second nucleic acid sequence encoding a transcription factor as defined in the first aspect of the invention.

The nucleic acid construct may have the following structure:
CAR-coexpr-TF; or
TF-coexpr-CAR
in which:
CAR is a nucleic acid sequence encoding the CAR;
coexpr is a nucleic acid sequence enabling co-expression of the CAR and the transcription factor; and
TF is a nucleic acid sequence encoding the transcription factor.

The nucleic acid sequence “coexpr” may encode a sequence comprising a self-cleaving peptide.

In a third aspect, the present invention provides a kit of nucleic acid sequences which comprise a first nucleic acid sequence encoding a Chimeric Antigen Receptor (CAR) and a second nucleic acid sequence encoding a transcription factor as defined in the first aspect of the invention.

In a fourth aspect, the present invention provides a vector which comprises a nucleic acid construct according to the second aspect of the invention.

In a fifth aspect, the present invention provides a kit of vectors which comprises a first vector which comprises a first nucleic acid sequence encoding a Chimeric Antigen Receptor (CAR); and a second vector which comprises a second nucleic acid sequence encoding a transcription factor as defined in the first aspect of the invention.

In a sixth aspect, the present invention provides a method for making a cell according to the first aspect of the invention, which comprises the step of introducing: a nucleic acid construct according to the second aspect of the invention, a kit of nucleic acid sequences according to the third aspect of the invention, a vector according to the fourth aspect of the invention, or a kit of vectors according to the fifth aspect of the invention, into a cell.

The cell may be from a sample isolated from a subject.

In a seventh aspect, the present invention provides a pharmaceutical composition comprising a plurality of cells according to the first aspect of the invention.

In an eighth aspect, the present invention provides a method for treating and/or preventing a disease, which comprises the step of administering a pharmaceutical composition according to the seventh aspect of the invention to a subject.

The method may comprise the following steps:
(i) isolation of a cell-containing sample from a subject;
(ii) transduction or transfection of the cells with: a nucleic acid construct according to the second aspect of the invention, a kit of nucleic acid sequences according to the third aspect of the invention, a vector according to the fourth aspect of the invention, or a kit of vectors according to the fifth aspect of the invention; and
(iii) administering the cells from (ii) to the subject.

In a ninth aspect, the present invention provides a pharmaceutical composition according to the seventh aspect of the invention for use in treating and/or preventing a disease.

In a tenth aspect, there is provided the use of a cell according to the first aspect of the invention in the manufacture of a medicament for treating and/or preventing a disease.

With reference to the eighth, ninth and tenth aspect of the invention, the disease may be a cancer.

### DETAILED DESCRIPTION

### Chimeric Antigen Receptor (CAR)

The cell of the present invention comprises an exogenous nucleic acid molecule encoding a chimeric antigen receptor (CAR).

A classical CAR is a chimeric type I trans-membrane protein which connects an extracellular antigen-recognizing domain (binder) to an intracellular signalling domain (endodomain). The binder is typically a single-chain variable fragment (scFv) derived from a monoclonal antibody (mAb), but it can be based on other formats which comprise an antibody-like antigen binding site. A spacer domain is usually necessary to isolate the binder from the membrane and to allow it a suitable orientation. A common spacer domain used is the Fc of IgG1. More compact spacers can suffice e.g. the stalk from CD8α and even just the IgG1 hinge alone, depending on the antigen. A trans-membrane domain anchors the protein in the cell membrane and connects the spacer to the endodomain.

Early CAR designs had endodomains derived from the intracellular parts of either the γ chain of the FcεR1 or CD3ζ. Consequently, these first generation receptors transmitted immunological signal 1, which was sufficient to trigger T-cell killing of cognate target cells but failed to fully activate the T-cell to proliferate and survive. To overcome this limitation, compound endodomains have been constructed: fusion of the intracellular part of a T-cell co-stimulatory molecule to that of CD3ζ results in second generation receptors which can transmit an activating and co-stimulatory signal simultaneously after antigen recognition. The co-stimulatory domain most commonly used is that of CD28. This supplies the most potent co-stimulatory signal—namely immunological signal 2, which triggers T-cell proliferation. Some receptors have also been described which include TNF receptor family endodomains, such as the closely related OX40 and 41 BB which transmit survival signals. Even more potent third generation CARs have now been described which have endodomains capable of transmitting activation, proliferation and survival signals.

CAR-encoding nucleic acids may be transferred to T cells using, for example, retroviral vectors. Lentiviral vectors may be employed. In this way, a large number of cancer-specific T cells can be generated for adoptive cell transfer. When the CAR binds the target-antigen, this results in the transmission of an activating signal to the T-cell it is expressed on. Thus the CAR directs the specificity and cytotoxicity of the T cell towards tumour cells expressing the targeted antigen. CARs typically therefore comprise: (i) an antigen-binding domain; (ii) a spacer; (iii) a transmembrane domain; and (iii) an intracellular domain which comprises or associates with a signalling domain.

### Antigen Binding Domain

The antigen binding domain is the portion of the CAR which recognizes antigen. Numerous antigen-binding domains are known in the art, including those based on the antigen binding site of an antibody, antibody mimetics, and T-cell receptors. For example, the antigen-binding domain may comprise: a single-chain variable fragment (scFv) derived from a monoclonal antibody; a natural ligand of the target antigen; a peptide with sufficient affinity for the target; a single domain antibody; an artificial single binder such as a Darpin (designed ankyrin repeat protein); or a single-chain derived from a T-cell receptor.

The antigen binding domain may comprise a domain which is not based on the antigen binding site of an antibody. For example the antigen binding domain may comprise a domain based on a protein/peptide which is a soluble ligand for a tumour cell surface receptor (e.g. a soluble peptide such as a cytokine or a chemokine); or an extracellular domain of a membrane anchored ligand or a receptor for which the binding pair counterpart is expressed on the tumour cell.

The antigen binding domain may be based on a natural ligand of the antigen.

The antigen binding domain may comprise an affinity peptide from a combinatorial library or a de novo designed affinity protein/peptide.

### Spacer Domain

CARs comprise a spacer sequence to connect the antigen-binding domain with the transmembrane domain and spatially separate the antigen-binding domain from the endodomain. A flexible spacer allows the antigen-binding domain to orient in different directions to facilitate binding.

### Transmembrane Domain

The transmembrane domain is the sequence of the CAR that spans the membrane.

A transmembrane domain may be any protein structure which is thermodynamically stable in a membrane. This is typically an alpha helix comprising of several hydrophobic residues. The transmembrane domain of any transmembrane protein can be used to supply the transmembrane portion of the invention. The presence and span of a transmembrane domain of a protein can be determined by those skilled in the art using the TMHMM algorithm (http://www.cbs.dtu.dk/services/TMHMM-2.0/).

Further, given that the transmembrane domain of a protein is a relatively simple structure, i.e a polypeptide sequence predicted to form a hydrophobic alpha helix of sufficient length to span the membrane, an artificially designed TM domain may also be used (U.S. Pat. No. 7,052,906 B1 describes synthetic transmembrane components).

The transmembrane domain may be derived from CD28, which gives good receptor stability.

### Endodomain

The endodomain is the signal-transmission portion of the CAR. It may be part of or associate with the intracellular domain of the CAR. After antigen recognition, receptors cluster, native CD45 and CD148 are excluded from the synapse and a signal is transmitted to the cell. The most commonly used endodomain component is that of CD3-zeta which contains 3 ITAMs. This transmits an activation signal to the T cell after antigen is bound. CD3-zeta may not provide a fully competent activation signal and additional co-stimulatory signaling may be needed. For example, chimeric CD28 and OX40 can be used with CD3-Zeta to transmit a proliferative/survival signal, or all three can be used together.

The endodomain of the CAR or TanCAR of the present invention may comprise the CD28 endodomain and OX40 and CD3-Zeta endodomain.

The endodomain may comprise:
(i) an ITAM-containing endodomain, such as the endodomain from CD3 zeta; and/or
(ii) a co-stimulatory domain, such as the endodomain from CD28; and/or
(iii) a domain which transmits a survival signal, for example a TNF receptor family endodomain such as OX-40 or 4-1 BB.

A number of systems have been described in which the antigen recognition portion is on a separate molecule from the signal transmission portion, such as those described in WO015/150771; WO2016/124930 and WO2016/030691. The CAR expressed by the cell of the present invention may therefore comprise an antigen-binding component comprising an antigen-binding domain and a transmembrane domain; which is capable of interacting with a separate intracellular signalling component comprising a signalling domain. The cell of the invention may comprise a CAR signalling system comprising such an antigen-binding component and intracellular signalling component.

### Signal Peptide

The cell of the present invention may comprise a signal peptide so that when the CAR is expressed inside a cell, the nascent protein is directed to the endoplasmic reticulum and subsequently to the cell surface, where it is expressed.

The signal peptide may be at the amino terminus of the molecule.

The CAR of the invention may have the general formula:

Signal peptide—antigen binding domain—spacer domain—transmembrane domain—intracellular T cell signaling domain (endodomain).

### Transcription Factor

The cell of the present invention comprises an exogenous nucleic acid molecule encoding a transcription factor.

A transcription factor is a protein which controls the rate of transcription of genetic information from DNA to messenger RNA, by binding to a specific DNA sequence and regulate the expression of a gene which comprises or is adjacent to that sequence.

Transcription factors work by promoting (as an activator), or blocking (as a repressor) the recruitment of RNA polymerase.

Transcription factors contain at least one DNA-binding domain (DBD), which attaches to either an enhancer or promoter region of DNA. Depending on the transcription factor, the transcription of the adjacent gene is either up- or down-regulated. Transcription factors also contain a trans-activating domain (TAD), which has binding sites for other proteins such as transcription coregulators.

Transcription factors use a variety of mechanisms for the regulation of gene expression, including stabilizing or blocking the binding of RNA polymerase to DNA, or catalyzing the acetylation or deacetylation of histone proteins. The transcription factor may have histone acetyltransferase (HAT) activity, which acetylates histone proteins, weakening the association of DNA with histones and making the DNA more accessible to transcription, thereby up-regulating transcription. Alternatively the transcription factor may have histone deacetylase (HDAC) activity, which deacetylates histone proteins, strengthening the association of DNA with histones and making the DNA less accessible to transcription, thereby down-regulating transcription. Another mechanism by which they may function is by recruiting coactivator or corepressor proteins to the transcription factor DNA complex.

There are two mechanistic classes of transcription factors, general transcription factors or upstream transcription factors.

General transcription factors are involved in the formation of a preinitiation complex. The most common are abbreviated as TFIIA, TFIIB, TFIID, TFIIE, TFIIF, and TFIIH. They are ubiquitous and interact with the core promoter region surrounding the transcription start site(s) of all class II genes.

Upstream transcription factors are proteins that bind upstream of the initiation site to stimulate or repress transcription. These are synonymous with specific transcription factors, because they vary considerably depending on what recognition sequences are present in the proximity of the gene.

Some examples of specific transcription factors are given in the table below:

| | Structural | Recognition | |
|---|---|---|---|
| Factor | type | sequence | Binds as |
| SP1 | Zinc finger | 5′-GGGCGG-3′ | Monomer |
| AP-1 | Basic zipper | 5′-TGA(G/C)TCA-3′ | Dimer |
| C/EBP | Basic zipper | 5′-ATTGCGCAAT-3′ | Dimer |
| Heat | Basic zipper | 5′-XGAAX-3′ | Trimer |
| shock | | | |
| factor | | | |
| ATF/ | Basic zipper | 5′-TGACGTCA-3′ | Dimer |
| CREB | | | |
| c-Myc | Basic helix- | 5′-CACGTG-3′ | Dimer |
| | loop-helix | | |
| Oct-1 | Helix-turn- | 5′-ATGCAAAT-3′ | Monomer |
| | helix | | |
| NF-1 | Novel | 5′-TTGGCXXXXX | Dimer |
| | | GCCAA-3′ | |

Transcription factors are often classified based on the sequence similarity and hence the tertiary structure of their DNA-binding domains.

Transcription factors with basic domains include: leucine zipper factors (e.g. bZIP, c-Fos/c-Jun, CREB and Plant G-box binding factors); helix-loop-helix factors (e.g. Ubiquitous (class A) factors; myogenic transcription factors (MyoD); Achaete-Scute and Tal/Twist/Atonal/Hen); and helix-loop-helix/leucine zipper factors (e.g. bHLH-ZIP, c-Myc, NF-1 (A, B, C, X), RF-X (1, 2, 3, 4, 5, ANK) and bHSH).

Transcription factors with zinc-coordinating DNA-binding domains include the Cys4 zinc finger of nuclear receptor type such as steroid hormone receptors and thyroid hormone receptor-like factors; diverse Cys4 zinc fingers such as GATA-Factors; Cys2His2 zinc finger domains, such as ubiquitous factors, including TFIIIA, Sp1; developmental/cell cycle regulators, including Kroppel; large factors with NF-6B-like binding properties; Cys6 cysteine-zinc cluster and zinc fingers of alternating composition.

Transcription factors with helix-turn-helix domains include those with homeodomains; paired box; fork head/winged helix; heat shock factors; tryptophan clusters; and TEAs (transcriptional enhancer factor) domain such as TEAD1, TEAD2, TEAD3, TEAD4.

Finally there are beta-scaffold factors with minor groove contacts including the RHR (Rel homology region) class; STAT; p53; MADS box; beta-Barrel alpha-helix transcription factors; TATA binding proteins; HMG-box; Heteromeric CCAAT factors; Grainyhead; Cold-shock domain factors; and Runt.

The transcription factor of the present invention may be constitutively active or conditionally active, i.e. requiring activation.

The transcription factor may be naturally occurring or artificial.

### Repression of T-Cell Differentiation

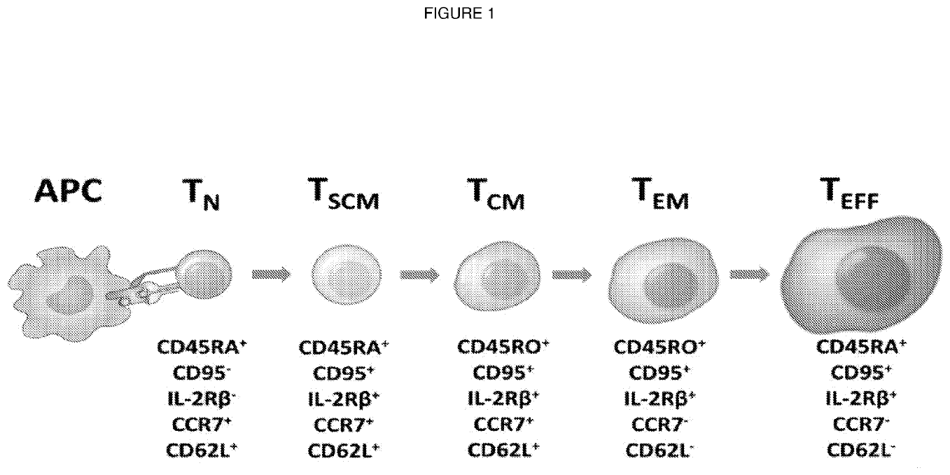

Following activation, T-cells differentiate into a variety of different T-cell subtypes, as shown in FIG. 1. The present inventors have shown that CAR T-cell persistence and engraftment in a subject is related to the proportion of naïve, central memory and stem-cell memory T-cells administered to the subject.

The cells of the invention may comprise an exogenous nucleic molecule encoding a transcription factor which effectively increases the proportion of naïve, central memory and/or stem-cell memory T cells in the composition for administration to a patient.

The transcription factor may, for example be a central memory repressing transcription factor such as BCL6 or BACH2. Central memory repressors inhibit the differentiation of T cells to effector memory cells, so that they remain as one of the less differentiated T-cell subtypes, such a naïve and stem cell memory T-cells. They block or reduce the rate of differentiation of T cells through the various stages shown in FIG. 1, biasing the T-cell population towards a more naïve phenotype.

Alternatively that transcription factor may be an effector memory repressing transcription factor such as BLIMP-1.

### BCL6

B-cell lymphoma protein (BCL6) is an evolutionarily conserved zinc finger transcription factor which contains an N-terminal POZ/BTB domain. BCL6 acts as a sequence-specific repressor of transcription, and has been shown to modulate the STAT-dependent Interleukin 4 (IL-4) responses of B cells. It interacts with several corepressor complexes to inhibit transcription.

The amino acid sequence of BCL6 is available from UniProt under accession No. P41182 and is shown as SEQ ID No. 1 below.

BCL6
SEQ ID No. 1
MASPADSCIQFTRHASDVLLNLNRLRSRDILTDVVIVVSREQFRAHKTVL
MACSGLFYSIFTDQLKCNLSVINLDPEINPEGFCILLDFMYTSRLNLREG
NIMAVMATAMYLQMEHVVDTCRKFIKASEAEMVSAIKPPREEFLNSRMLM
PQDIMAYRGREVVENNLPLRSAPGCESRAFAPSLYSGLSTPPASYSMYSH
LPVSSLLFSDEEFRDVRMPVANPFPKERALPCDSARPVPGEYSRPTLEVS
PNVCHSNIYSPKETIPEEARSDMHYSVAEGLKPAAPSARNAPYFPCDKAS
KEEERPSSEDEIALHFEPPNAPLNRKGLVSPQSPQKSDCQPNSPTESCSS
KNACILQASGSPPAKSPTDPKACNWKKYKFIVLNSLNQNAKPEGPEQAEL
GRLSPRAYTAPPACQPPMEPENLDLQSPTKLSASGEDSTIPQASRLNNIV
NRSMTGSPRSSSESHSPLYMHPPKCTSCGSQSPQHAEMCLHTAGPTFPEE
MGETQSEYSDSSCENGAFFCNECDCRFSEEASLKRHTLQTHSDKPYKCDR
CQASFRYKGNLASHKTVHTGEKPYRCNICGAQFNRPANLKTHTRIHSGEK
PYKCETCGARFVQVAHLRAHVLIHTGEKPYPCEICGTRFRHLQTLKSHLR
IHTGEKPYHCEKCNLHFRHKSQLRLHLRQKHGAITNTKVQYRVSATDLPP
ELPKAC

BCL6 comprises six zinc fingers at the following amino acid positions: 518-541, 546-568, 574-596, 602-624, 630-652, 658-681.

### BACH2

The broad complex and cap‘n’collar homology (Bach)2 protein, also known as bric-a-brac and tramtrack, and is a 92 kDa transcriptional factor. Via a basic leucine zipper domain, it heterodimerizes with proteins of the musculoaponeurotic fibrosarcoma (Maf) family. The Bach2 gene locus resides in a Super Enhancer (SE), and regulates the expression of the SE-regulated genes. SEs are crucial for cell-lineage gene expression. In T-cells, the majority of SE-regulated genes are cytokines and cytokine receptor genes. Bach2 is a predominant gene associated with SE in all T-cell lineages.

The Bach2 protein consists of 72 phosphorylation sites. Of those sites, Ser-335 consists of the consensus sequence of Akt targets (RXRXX(S/T)X). Eleven sites (Ser-260, Ser-314, Thr-318, Thr-321, Ser-336, Ser-408, Thr-442, Ser-509, Ser-535, Ser-547, and Ser-718) bear the consensus sequence of mTOR targets (proline at +1 position). Substitution of Ser-535 and Ser-509 to Ala increases the nuclear localisation of Bach2, and augments the downregulation of its target genes.

The site Ser-520 has been identified as an Akt substrate for phosphorylation. Substitution of Ser-520 to Ala also increases the repressor capacity of Bach2. eGFP fusion to the WT or mutated Bach2 revealed an augmented nuclear localisation of S520A Bach2. The phosphorylation of Bach2 upon T-cell activation leads to Bach2 sequestration in the cytoplasm. Mutations at the phosphorylation site render Bach2 resistant to such sequestration, and thus its localisation to the nucleus is increased.

The cell of the present invention may comprise an exogenous nucleic acid molecule which expresses a variant of Bach2 which has increased nuclear localisation compared to the wild type protein. The variant may have a mutation at Ser-535, Ser-520 or Ser-509 with reference to the sequence shown as SEQ ID No. 2. The mutation may be a substitution, such as a Ser to Ala substitution.

Bach2 binds on the consensus motif (5′-TGA(C/G)TCAGC-3′), which is part of the motif (5′-TGA(C/G)TCA-3′) recognised by the AP-1 family. AP-1 family of transcription factors is involved in inducing the expression of genes downstream of TCR activation. The AP-1 transcription factor family includes c-Jun, JunB and c-Fos. AP-1 factors are phosphorylated upon TCR activation, and subsequently regulate genes involved in effector differentiation. Bach2 represses the activation of those genes, by competing with AP-1 for binding on overlapping motifs.

The expression of Bach2 mRNA is high in naïve CD8 T-cells, and is gradually downregulated in central memory (CD62L+KLRG1−), effector (CD62L−KLRG1−) and terminally differentiated effector (CD62L−KLRG1+) cells. Deficiency of Bach2 leads to terminally differentiated T-cells, and increases apoptosis.

The amino acid sequence of Bac2 is available from UniProt under accession No. Q9BYV9 and is shown as SEQ ID No. 2 below.

Bach-2 wild type
SEQ ID No. 2
MSVDEKPDSPMYVYESTVHCTNILLGLNDQRKKDILCDVTLIVERKEFRA
HRAVLAACSEYFWQALVGQTKNDLVVSLPEEVTARGFGPLLQFAYTAKLL
LSRENIREVIRCAEFLRMHNLEDSCFSFLQTQLLNSEDGLFVCRKDAACQ
RPHEDCENSAGEEEDEEEETMDSETAKMACPRDQMLPEPISFEAAAIPVA
EKEEALLPEPDVPTDTKESSEKDALTQYPRYKKYQLACTKNVYNASSHST
SGFASTFREDNSSNSLKPGLARGQIKSEPPSEENEEESITLCLSGDEPDA
KDRAGDVEMDRKQPSPAPTPTAPAGAACLERSRSVASPSCLRSLFSITKS
VELSGLPSTSQQHFARSPACPFDKGITQGDLKTDYTPFTGNYGQPHVGQK
EVSNFTMGSPLRGPGLEALCKQEGELDRRSVIFSSSACDQVSTSVHSYSG
VSSLDKDLSEPVPKGLWVGAGQSLPSSQAYSHGGLMADHLPGRMRPNTSC
PVPIKVCPRSPPLETRTRTSSSCSSYSYAEDGSGGSPCSLPLCEFSSSPC
SQGARFLATEHQEPGLMGDGMYNQVRPQIKCEQSYGTNSSDESGSFSEAD
SESCPVQDRGQEVKLPFPVDQITDLPRNDFQMMIKMHKLTSEQLEFIHDV
RRRSKNRIAAQRCRKRKLDCIQNLECEIRKLVCEKEKLLSERNQLKACMG
ELLDNFSCLSQEVCRDIQSPEQIQALHRYCPVLRPMDLPTASSINPAPLG
AEQNIAASQCAVGENVPCCLEPGAAPPGPPWAPSNTSENCTSGRRLEGTD
PGTFSERGPPLEPRSQTVTVDFCQEMTDKCTTDEQPRKDYT

A mutant Bach2 sequence which has an S to A substitution at position 520 is shown as SEQ ID No. 3. The S520A substitution is in bold and underlined.

### BLIMP-1

S520A Bach2 mutant (insensitive to AKT)
SEQ ID No. 3
MSVDEKPDSPMYVYESTVHCTNILLGLNDQRKKDILCDVTLIVERKEFRA
HRAVLAACSEYFWQALVGQTKNDLVVSLPEEVTARGFGPLLQFAYTAKLL
LSRENIREVIRCAEFLRMHNLEDSCFSFLQTQLLNSEDGLFVCRKDAACQ
RPHEDCENSAGEEEDEEEETMDSETAKMACPRDQMLPEPISFEAAAIPVA
EKEEALLPEPDVPTDTKESSEKDALTQYPRYKKYQLACTKNVYNASSHST
SGFASTFREDNSSNSLKPGLARGQIKSEPPSEENEEESITLCLSGDEPDA
KDRAGDVEMDRKQPSPAPTPTAPAGAACLERSRSVASPSCLRSLFSITKS
VELSGLPSTSQQHFARSPACPFDKGITQGDLKTDYTPFTGNYGQPHVGQK
EVSNFTMGSPLRGPGLEALCKQEGELDRRSVIFSSSACDQVSTSVHSYSG
VSSLDKDLSEPVPKGLWVGAGQSLPSSQAYSHGGLMADHLPGRMRPNTSC
PVPIKVCPRSPPLETRTRTSASCSSYSYAEDGSGGSPCSLPLCEFSSSPC
SQGARFLATEHQEPGLMGDGMYNQVRPQIKCEQSYGTNSSDESGSFSEAD
SESCPVQDRGQEVKLPFPVDQITDLPRNDFQMMIKMHKLTSEQLEFIHDV
RRRSKNRIAAQRCRKRKLDCIQNLECEIRKLVCEKEKLLSERNQLKACMG
ELLDNFSCLSQEVCRDIQSPEQIQALHRYCPVLRPMDLPTASSINPAPLG
AEQNIAASQCAVGENVPCCLEPGAAPPGPPWAPSNTSENCTSGRRLEGTD
PGTFSERGPPLEPRSQTVTVDFCQEMTDKCTTDEQPRKDYT

B-lymphocyte-induced maturation protein 1 (BLIMPI) acts as a repressor of beta-interferon (β-IFN) gene expression. The protein binds specifically to the PRDI (positive regulatory domain I element) of the β-IFN gene promoter.

The increased expression of the Blimp-1 protein in B lymphocytes, T lymphocytes, NK cell and other immune system cells leads to an immune response through proliferation and differentiation of antibody secreting plasma cells. Blimp-1 is also considered a ‘master regulator’ of hematopoietic stem cells.

BLIMP-1 is involved in controlling the terminal differentiation of antibody-secreting cells (ASCs) and has an important role in maintaining the homeostasis of effector T cells.

The amino acid sequence of BLIMP-1 is available from UniProt under accession No. 075626 and is shown as SEQ ID No. 4 below.

BLIMP-1
SEQ ID No. 4
MLDICLEKRVGTTLAAPKCNSSTVRFQGLAEGTKGTMKMDMEDADMTLWT
EAEFEEKCTYIVNDHPWDSGADGGTSVQAEASLPRNLLFKYATNSEEVIG
VMSKEYIPKGTRFGPLIGEIYTNDTVPKNANRKYFWRIYSRGELHHFIDG
FNEEKSNWMRYVNPAHSPREQNLAACQNGMNIYFYTIKPIPANQELLVWY
CRDFAERLHYPYPGELTMMNLTQTQSSLKQPSTEKNELCPKNVPKREYSV
KEILKLDSNPSKGKDLYRSNISPLTSEKDLDDFRRRGSPEMPFYPRVVYP
IRAPLPEDFLKASLAYGIERPTYITRSPIPSSTTPSPSARSSPDQSLKSS
SPHSSPGNTVSPVGPGSQEHRDSYAYLNASYGTEGLGSYPGYAPLPHLPP
AFIPSYNAHYPKFLLPPYGMNCNGLSAVSSMNGINNFGLFPRLCPVYSNL
LGGGSLPHPMLNPTSLPSSLPSDGARRLLQPEHPREVLVPAPHSAFSFTG
AAASMKDKACSPTSGSPTAGTAATAEHVVQPKATSAAMAAPSSDEAMNLI
KNKRNMTGYKTLPYPLKKQNGKIKYECNVCAKTFGQLSNLKVHLRVHSGE
RPFKCQTCNKGFTQLAHLQKHYLVHTGEKPHECQVCHKRFSSTSNLKTHL
RLHSGEKPYQCKVCPAKFTQFVHLKLHKRLHTRERPHKCSQCHKNYIHLC
SLKVHLKGNCAAAPAPGLPLEDLTRINEEIEKFDISDNADRLEDVEDDIS
VISVVEKEILAVVRKEKEETGLKVSLQRNMGNGLLSSGCSLYESSDLPLM
KLPPSNPLPLVPVKVKQETVEPMDP

### FOXO1

Forkhead box protein O1 (FOXO1) also known as forkhead in rhabdomyosarcoma is a protein that in humans is encoded by the FOXO1 gene. FOXO1 is a transcription factor that plays important roles in regulation of gluconeogenesis and glycogenolysis by insulin signaling, and is also central to the decision for a preadipocyte to commit to adipogenesis. It is primarily regulated through phosphorylation on multiple residues; its transcriptional activity is dependent on its phosphorylation state.

The amino acid sequence of FOXO1 is available from UniProt under accession No. Q12778 and is shown as SEQ ID No. 5 below.

FOXO1
SEQ ID No. 5
AEAPQVVEIDPDFEPLPRPRSCTWPLPRPEFSQSNSATSSPAPSGSAAAN
PDAAAGLPSASAAAVSADFMSNLSLLEESEDFPQAPGSVAAAVAAAAAAA
ATGGLCGDFQGPEAGCLHPAPPQPPPPGPLSQHPPVPPAAAGPLAGQPRK
SSSSRRNAWGNLSYADLITKAIESSAEKRLTLSQIYEWMVKSVPYFKDKG
DSNSSAGWKNSIRHNLSLHSKFIRVQNEGIGKSSWWMLNPEGGKSGKSPR
RRAASMDNNSKFAKSRSRAAKKKASLQSGQEGAGDSPGSQFSKWPASPGS
HSNDDFDNWSTFRPRTSSNASTISGRLSPIMTEQDDLGEGDVHSMVYPPS
AAKMASTLPSLSEISNPENMENLLDNLNLLSSPTSLTVSTQSSPGTMMQQ
TPCYSFAPPNTSLNSPSPNYQKYTYGQSSMSPLPQMPIQTLQDNKSSYGG
MSQYNCAPGLLKELLTSDSPPHNDIMTPVDPGVAQPNSRVLGQNVMMGPN
SVMSTYGSQASHNKMMNPSSHTHPGHAQQTSAVNGRPLPHTVSTMPHTSG
MNRLTQVKTPVQVPLPHPMQMSALGGYSSVSSCNGYGRMGLLHQEKLPSD
LDGMFIERLDCDMESIIRNDLMDGDTLDFNFDNVLPNQSFPHSVKTTTHS
WVSG

### EOMES

EOMES, also known as Eomesodermin and T-box brain protein 2 (Tbr2) is a protein that in humans is encoded by the EOMES gene. It is a member of a conserved protein family that shares a common DNA-binding domain, the T-box. T-box genes encode transcription factors, which control gene expression, involved in the regulation of developmental processes. Eomes itself controls regulation of radial glia, as well as other related cells. Eomes has also been found to have a role in immune response, and there exists some loose evidence for its connections in other systems.

The amino acid sequence of EOMES is available from UniProt under accession No. 095936 and is shown as SEQ ID No. 6 below.

EOMES
SEQ ID No. 6
QLGEQLLVSSVNLPGAHFYPLESARGGSGGSAGHLPSAAPSPQKLDLDKA
SKKFSGSLSCEAVSGEPAAASAGAPAAMLSDTDAGDAFASAAAVAKPGPP
DGRKGSPCGEEELPSAAAAAAAAAAAAAATARYSMDSLSSERYYLQSPGP
QGSELAAPCSLFPYQAAAGAPHGPVYPAPNGARYPYGSMLPPGGFPAAVC
PPGRAQFGPGAGAGSGAGGSSGGGGGPGTYQYSQGAPLYGPYPGAAAAGS
CGGLGGLGVPGSGFRAHVYLCNRPLWLKFHRHQTEMIITKQGRRMFPFLS
FNINGLNPTAHYNVFVEVVLADPNHWRFQGGKWVTCGKADNNMQGNKMYV
HPESPNTGSHWMRQEISFGKLKLTNNKGANNNNTQMIVLQSLHKYQPRLH
IVEVTEDGVEDLNEPSKTQTFTFSETQFIAVTAYQNTDITQLKIDHNPFA
KGFRDNYDSSHQIVPGGRYGVQSFFPEPFVNTLPQARYYNGERTVPQTNG
LLSPQQSEEVANPPQRWLVTPVQQPGTNKLDISSYESEYTSSTLLPYGIK
SLPLQTSHALGYYPDPTFPAMAGWGGRGSYQRKMAAGLPWTSRTSPTVFS
EDQLSKEKVKEEIGSSWIETPPSIKSLDSNDSGVYTSACKRRRLSPSNSS
NENSPSIKCEDINAEEYSKDTSKGMGGYYAFYTT

### RUNX3

Runx3 or Runt-related transcription factor 3 is a member of the runt domain-containing family of transcription factors. A heterodimer of this protein and a beta subunit forms a complex that binds to the core DNA sequence 5′-YGYGGT-3′ found in a number of enhancers and promoters, and can either activate or suppress transcription. It also interacts with other transcription factors. It functions as a tumor suppressor, and the gene is frequently deleted or transcriptionally silenced in cancer. Multiple transcript variants encoding different isoforms have been found for this gene.

The amino acid sequence of RUNX3 is available from UniProt under accession No. Q13761 and is shown below as SEQ ID No. 7.

RUNX3
SEQ ID No. 7
MRIPVDPSTSRRFTPPSPAFPCGGGGGKMGENSGALSAQAAVGPGGRARP
EVRSMVDVLADHAGELVRTDSPNFLCSVLPSHWRCNKTLPVAFKVVALGD
VPDGTVVTVMAGNDENYSAELRNASAVMKNQVARFNDLRFVGRSGRGKSF
TLTITVFTNPTQVATYHRAIKVTVDGPREPRRHRQKLEDQTKPFPDRFGD
LERLRMRVTPSTPSPRGSLSTTSHFSSQPQTPIQGTSELNPFSDPRQFDR
SFPTLPTLTESRFPDPRMHYPGAMSAAFPYSATPSGTSISSLSVAGMPAT
SRFHHTYLPPPYPGAPQNQSGPFQANPSPYHLYYGTSSGSYQFSMVAGSS
SGGDRSPTRMLASCTSSAASVAAGNLMNPSLGGQSDGVEADGSHSNSPTA
LSTPGRMDEAVWRPYPAAKRVKLD

### CBF BETA

Core-binding factor subunit beta (CBF beta) is the beta subunit of a heterodimeric core-binding transcription factor belonging to the PEBP2/CBF transcription factor family which master-regulates a host of genes specific to hematopoiesis (e.g., RUNX1) and osteogenesis (e.g., RUNX2). The beta subunit is a non-DNA binding regulatory subunit; it allosterically enhances DNA binding by the alpha subunit as the complex binds to the core site of various enhancers and promoters, including murine leukemia virus, polyomavirus enhancer, T-cell receptor enhancers and GM-CSF promoters. Alternative splicing generates two mRNA variants, each encoding a distinct carboxyl terminus.

The amino acid sequence of CBF beta is available from UniProt under accession No. Q13951 and is shown below as SEQ ID No. 8.

CBF beta
SEQ ID No. 8
MPRVVPDQRSKFENEEFFRKLSRECEIKYTGFRDRPHEERQARFQNACRD
GRSEIAFVATGTNLSLQFFPASWQGEQRQTPSREYVDLEREAGKVYLKAP
MILNGVCVIWKGWIDLQRLDGMGCLEFDEERAQQEDALAQQAFEEARRRT
REFEDRDRSHREEMEARRQQDPSPGSNLGGGDDLKLR

### Exogenous Nucleic Acid Molecule

The present invention provides a cell which comprises an exogenous nucleic acid molecule encoding a transcription factor. The word “exogenous” means that the nucleic acid molecule is made by recombinant means and is introduced into the cell by way of a vector. The cell is engineered to contain the nucleic acid molecule and to express (or over-express) the transcription factor.

### Nucleic Acid

As used herein, the terms “polynucleotide”, “nucleotide”, and “nucleic acid” are intended to be synonymous with each other.

It will be understood by a skilled person that numerous different polynucleotides and nucleic acids can encode the same polypeptide as a result of the degeneracy of the genetic code. In addition, it is to be understood that skilled persons may, using routine techniques, make nucleotide substitutions that do not affect the polypeptide sequence encoded by the polynucleotides described here to reflect the codon usage of any particular host organism in which the polypeptides are to be expressed.

Nucleic acids according to the invention may comprise DNA or RNA. They may be single-stranded or double-stranded. They may also be polynucleotides which include within them synthetic or modified nucleotides. A number of different types of modification to oligonucleotides are known in the art. These include methylphosphonate and phosphorothioate backbones, addition of acridine or polylysine chains at the 3′ and/or 5′ ends of the molecule. For the purposes of the use as described herein, it is to be understood that the polynucleotides may be modified by any method available in the art. Such modifications may be carried out in order to enhance the in vivo activity or life span of polynucleotides of interest.

The terms “variant”, “homologue” or “derivative” in relation to a nucleotide sequence include any substitution of, variation of, modification of, replacement of, deletion of or addition of one (or more) nucleic acid from or to the sequence.

### Nucleic Acid Construct

The present invention provides a nucleic acid construct which comprises a first nucleic acid sequence encoding a Chimeric Antigen Receptor (CAR) and a second nucleic acid sequence encoding a transcription factor.

The nucleic acid construct may also comprise a nucleic acid sequence enabling expression of two or more proteins. For example, it may comprise a sequence encoding a cleavage site between the two nucleic acid sequences. The cleavage site may be self-cleaving, such that when the nascent polypeptide is produced, it is immediately cleaved into the two proteins without the need for any external cleavage activity.

Various self-cleaving sites are known, including the Foot-and-Mouth disease virus (FMDV) 2a self-cleaving peptide, which has the sequence:

SEQ ID NO: 9
RAEGRGSLLTCGDVEENPGP
or
SEQ ID NO: 10
QCTNYALLKLAGDVESNPGP

The co-expressing sequence may alternatively be an internal ribosome entry sequence (IRES) or an internal promoter.

### Vector

The present invention also provides a vector, or kit of vectors which comprises one or more nucleic acid sequence(s) or construct(s) according to the present invention. Such a vector may be used to introduce the nucleic acid sequence(s) or construct(s) into a host cell so that it expresses the proteins encoded by the nucleic acid sequence or construct.

The vector may, for example, be a plasmid or a viral vector, such as a retroviral vector or a lentiviral vector, or a transposon based vector or synthetic mRNA.

The vector may be capable of transfecting or transducing a T cell.

### Cell

The present invention provides a cell which co-expresses a CAR a transcription factor.

The cell may be a cytolytic immune cell.

Cytolytic immune cells can be T cells or T lymphocytes which are a type of lymphocyte that play a central role in cell-mediated immunity. They can be distinguished from other lymphocytes, such as B cells and natural killer cells (NK cells), by the presence of a T-cell receptor (TCR) on the cell surface. There are various types of T cell, as summarised below.

Helper T helper cells (TH cells) assist other white blood cells in immunologic processes, including maturation of B cells into plasma cells and memory B cells, and activation of cytotoxic T cells and macrophages. TH cells express CD4 on their surface. TH cells become activated when they are presented with peptide antigens by MHC class II molecules on the surface of antigen presenting cells (APCs). These cells can differentiate into one of several subtypes, including TH1, TH2, TH3, TH17, Th9, or TFH, which secrete different cytokines to facilitate different types of immune responses.

Cytolytic T cells (TC cells, or CTLs) destroy virally infected cells and tumor cells, and are also implicated in transplant rejection. CTLs express the CD8 at their surface. These cells recognize their targets by binding to antigen associated with MHC class I, which is present on the surface of all nucleated cells. Through IL-10, adenosine and other molecules secreted by regulatory T cells, the CD8+ cells can be inactivated to an anergic state, which prevent autoimmune diseases such as experimental autoimmune encephalomyelitis.

Memory T cells are a subset of antigen-specific T cells that persist long-term after an infection has resolved. They quickly expand to large numbers of effector T cells upon re-exposure to their cognate antigen, thus providing the immune system with “memory” against past infections. Memory T cells comprise three subtypes: central memory T cells (TCM cells) and two types of effector memory T cells (TEM cells and TEMRA cells). Memory cells may be either CD4+ or CD8+. Memory T cells typically express the cell surface protein CD45RO.

Regulatory T cells (Treg cells), formerly known as suppressor T cells, are crucial for the maintenance of immunological tolerance. Their major role is to shut down T cell-mediated immunity toward the end of an immune reaction and to suppress auto-reactive T cells that escaped the process of negative selection in the thymus.

Two major classes of CD4+ Treg cells have been described—naturally occurring Treg cells and adaptive Treg cells.

Naturally occurring Treg cells (also known as CD4+CD25+FoxP3+Treg cells) arise in the thymus and have been linked to interactions between developing T cells with both myeloid (CD11c+) and plasmacytoid (CD123+) dendritic cells that have been activated with TSLP. Naturally occurring Treg cells can be distinguished from other T cells by the presence of an intracellular molecule called FoxP3. Mutations of the FOXP3 gene can prevent regulatory T cell development, causing the fatal autoimmune disease IPEX.

Adaptive Treg cells (also known as Tr1 cells or Th3 cells) may originate during a normal immune response.

Natural Killer Cells (or NK cells) are a type of cytolytic cell which form part of the innate immune system. NK cells provide rapid responses to innate signals from virally infected cells in an MHC independent manner.

NK cells (belonging to the group of innate lymphoid cells) are defined as large granular lymphocytes (LGL) and constitute the third kind of cells differentiated from the common lymphoid progenitor generating B and T lymphocytes. NK cells are known to differentiate and mature in the bone marrow, lymph node, spleen, tonsils and thymus where they then enter into the circulation.

The cells of the invention may be any of the cell types mentioned above.

Where the transcription factor reduces or inhibits T-cell differentiation or exhaustion, the cell may preferentially be one of the following T-cell subtypes: naïve T cell; stem cell memory T cell; and central memory T cells.

Cells of the invention may either be created ex vivo either from a patient's own peripheral blood (1st party), or in the setting of a haematopoietic stem cell transplant from donor peripheral blood (2nd party), or peripheral blood from an unconnected donor (3rd party).

Alternatively, the cells may be derived from ex vivo differentiation of inducible progenitor cells or embryonic progenitor cells to, for example, T cells. Alternatively, an immortalized cell line which retains its lytic function and could act as a therapeutic may be used.

In all these embodiments, cells may generated by introducing DNA or RNA coding for the CAR and transcription factor by one of many means including transduction with a viral vector, transfection with DNA or RNA.

The cell of the invention may be an ex vivo cell from a subject. The cell may be from a peripheral blood mononuclear cell (PBMC) sample. Cells may be activated and/or expanded prior to being transduced with nucleic acid sequence or construct of the invention, for example by treatment with an anti-CD3 monoclonal antibody.

The cell of the invention may be made by:
(i) isolation of a cell-containing sample from a subject or other sources listed above; and
(ii) transduction or transfection of the cells with a nucleic acid sequence or construct according to the invention.

### Composition

The present invention also relates to a pharmaceutical composition containing a plurality of cells of the invention. The pharmaceutical composition may additionally comprise a pharmaceutically acceptable carrier, diluent or excipient. The pharmaceutical composition may optionally comprise one or more further pharmaceutically active polypeptides and/or compounds. Such a formulation may, for example, be in a form suitable for intravenous infusion.

### Method of Treatment

The cells of the present invention may be capable of killing target cells, such as cancer cells.

The cells of the present invention may be used for the treatment of an infection, such as a viral infection.

The cells of the invention may also be used for the control of pathogenic immune responses, for example in autoimmune diseases, allergies and graft-vs-host rejection.

The cells of the invention may be used for the treatment of a cancerous disease, such as bladder cancer, breast cancer, colon cancer, endometrial cancer, kidney cancer (renal cell), leukemia, lung cancer, melanoma, non-Hodgkin lymphoma, pancreatic cancer, prostate cancer and thyroid cancer.

The cells of the invention may be used to treat: cancers of the oral cavity and pharynx which includes cancer of the tongue, mouth and pharynx; cancers of the digestive system which includes oesophageal, gastric and colorectal cancers; cancers of the liver and biliary tree which includes hepatocellular carcinomas and cholangiocarcinomas; cancers of the respiratory system which includes bronchogenic cancers and cancers of the larynx; cancers of bone and joints which includes osteosarcoma; cancers of the skin which includes melanoma; breast cancer; cancers of the genital tract which include uterine, ovarian and cervical cancer in women, prostate and testicular cancer in men; cancers of the renal tract which include renal cell carcinoma and transitional cell carcinomas of the utterers or bladder; brain cancers including gliomas, glioblastoma multiforme and medullobastomas; cancers of the endocrine system including thyroid cancer, adrenal carcinoma and cancers associated with multiple endocrine neoplasm syndromes; lymphomas including Hodgkin's lymphoma and non-Hodgkin lymphoma; Multiple Myeloma and plasmacytomas; leukaemias both acute and chronic, myeloid or lymphoid; and cancers of other and unspecified sites including neuroblastoma.

The invention will now be further described by way of Examples, which are meant to serve to assist one of ordinary skill in the art in carrying out the invention and are not intended in any way to limit the scope of the invention.

### EXAMPLES

### Example 1—Chimeric Antigen Receptor (CAR and Transcription Factor (TF) Co-Expression

A bicistronic construct was expressed in BW5 T cells as a single transcript which self-cleaves at the 2A site to yield a chimeric antigen receptor (CAR); and a transcription factor (TF). Control constructs were also generated which lack the 2A site and the transcription factor (“CAR-only”) or lack the CAR and 2A site (“TF-only”).

The CAR was an anti-CD19 CAR comprising an endodomain derived from CD3 zeta and from the co-stimulatory receptor 41 BB.

Constructs were tested comprising the transcription factors shown in the following table:

| | Transcription |
|---|---|
| Transcription factor type | factor |
| Central memory transcription factors | EOMES |
| | FOXO1 |
| | Runx3 and beta |
| | catenin |
| Central memory repressors | BACH2 |

### Example 2—Phenotype Assays

The expression of various CARs on the surface of T cells can influence the memory status of those T cells in the absence of the CAR antigen. In addition, binding of the CAR to its cognate antigen activates the T cells and causes further differentiation from a more naïve central memory phenotype to a more differentiated effector memory/effector phenotype. Expression of the appropriate transcription factor/repressor is expected to prevent this CAR-mediated differentiation to varying degrees.

T cells expressing the various CAR-TF combinations, together with the relevant CAR-only and TF-only controls were co-cultured with CD19 positive SKOV3 target cells for 24 hours before recovering and culturing the T cells until day 7. The expression of the following memory markers was analysed by flow cytometry at day 0 of the co-culture and day 7, to see whether cells expressing factors that bias them towards central memory are more naïve post-transduction and remain more naïve upon stimulation with antigen-bearing target cells.

### Memory Markers—CCR7, CD45RA, CD62L, CD27

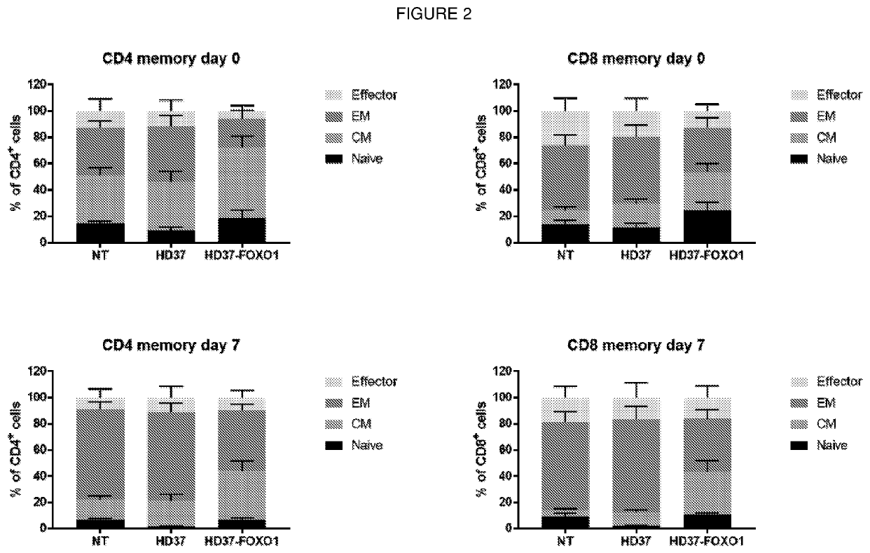

The data for FOXO1 are shown in FIG. 2. For both CD4+ and CD8+ subpopulations, the co-expression of FOXO1 with the CAR (HD37) gave a greater proportion of naïve and central memory cells (CM) at both day 0 and day 7. This indicates that FOXO1 biases the cells towards a naïve/central memory phenotype both post-transduction and following co-culture with target cells.

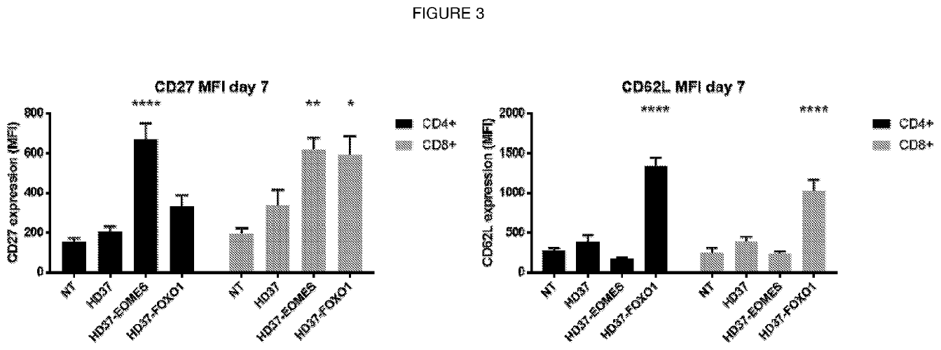

FIG. 3 shows CD27 and CD62L expression data 6 days after a 24 hour co-culture with target cells. The transcription factor EOMES caused significant upregulation of CD27 in both the CD4+ and CD8+ T cell subpopulations. FOXO1 caused upregulation of CD27, especially on CD8+ cells. The transcription factor FOXO1 caused significant upregulation of CD62L on both the CD4+ and CD8+ subpopulations. CD62L is a marker of naïve/central memory cells and memory phenotyping for FOX01 correlates with the CD62L levels: more naïve and memory cells. CD27 is a marker of everything other than fully differentiated effector cells, so it could be that the EOMES-expressing cells are predominantly a less differentiated effector memory sub-type which do not show significant up-regulation of CD62L.

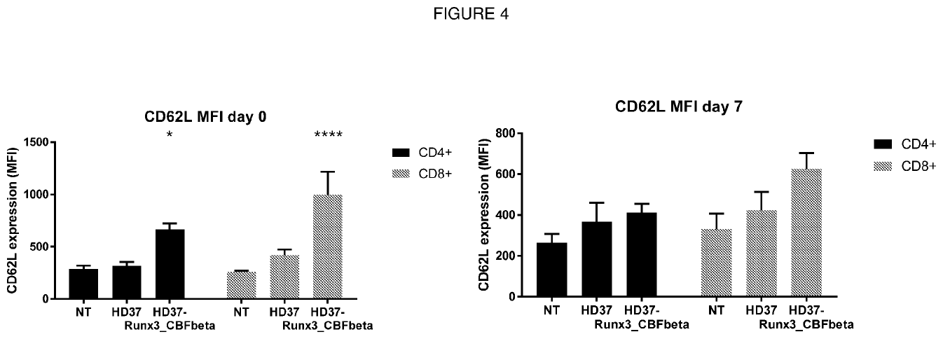

As shown in FIG. 4, the presence of both Runx3 and CBFbeta caused upregulation of CD62L after transduction (day 0) and 6 days after the 24 hour co-culture.

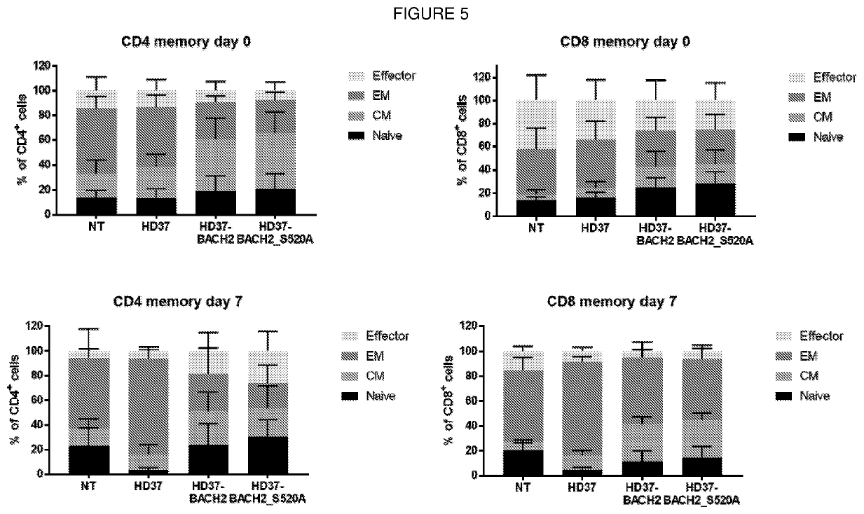

The data for BACH2 and the BACH2 mutant S520A are shown in FIG. 5. Both BACH2 and BACH2 S520A give an increase in the proportion of naïve and central memory cells (CM) at both day 0 and day 7.

In separate assays, T cells expressing the various CAR-TF combinations together with the relevant CAR-only were co-cultured with CD19 positive SupT1 target cells. The expression of the following exhaustion markers was analysed by flow cytometry at day 0 of the co-culture and days 2, 4, and 7, to see whether cells express “Exhaustion” markers to a lower degree upon stimulation.

### Exhaustion Markers—PD1, Tim3, Lag3

The cells were gated on CAR-expression (via RQR8 transduction marker) and various T cell and T-cell subset markers (CD3 and CD8) depending on the sub-population of interest.

### Example 3—Functional Assays

As cells progressively differentiate they acquire greater capacity to lyse target cells in vitro and secrete more IFN-γ but a have a lower capacity to proliferate. This study investigates whether biasing phenotype alters these capacities.

SupT1 cells (which are CD19 negative), are engineered to be CD19 positive giving target negative and positive cell lines. Transduced and non-transduced T-cells and T-cells transduced with the control constructs are challenged 1:1 with either SupT1 cells or SupT1.CD19 cells. Killing of target cells is analysed by FACS after 24 and 72 hours. Killing is also monitored using an Incucyte assay which involves co-culturing transduced T cells on a monolayer of fluorescently-labelled adherent cells expressing the respective CAR antigen. CAR-mediated cytotoxicity result in reduction in the number of fluorescent target cells which can be monitored on a continuous basis over time allowing measurement of the kinetics of CAR-mediated cytotoxicity.

In order to monitor cytokine release, supernatant is sampled 48 hours after challenge and cytokine bead arrays are used to assay for the following cytokines: IL2, IL4, IL6, IL10, TNF-a, IFN-g, GzmB.

In order to measure proliferation, the T-cells are labelled with the fluorescent dye Cell Trace Violet for 20 min. After labelling, a co-culture is set up at a ratio of 1:1 (target cells:transduced T-cells). CAR-mediated proliferation of the T cells results in a reduction in the Cell Trace violet fluorescence as the dye is divided between successive daughter cell generations. The extent to which this dilution has occurred, and therefore the degree of proliferation, can be measured by flow cytometry at days 4 and 7 of the co-culture.

All publications mentioned in the above specification are herein incorporated by reference. Various modifications and variations of the described methods and system of the invention will be apparent to those skilled in the art without departing from the scope and spirit of the invention. Although the invention has been described in connection with specific preferred embodiments, it should be understood that the invention as claimed should not be unduly limited to such specific embodiments. Indeed, various modifications of the described modes for carrying out the invention which are obvious to those skilled in molecular biology or related fields are intended to be within the scope of the following claims.

1. A cell which comprises a first exogenous nucleic acid molecule encoding a Chimeric Antigen Receptor (CAR) and a second exogenous nucleic acid molecule encoding a transcription factor.

2. A cell according to claim 1, wherein the transcription factor prevents or reduces differentiation and/or exhaustion of the cell.

3. A cell according to claim 1, wherein the transcription factor is an effector memory repressor.

4. A cell according to claim 3, wherein the transcription factor is BLIMP-1.

5. A cell according to claim 1, wherein the transcription factor is a central memory repressor.

6. A cell according to claim 5, wherein the transcription factor is BCL6 or Bach2.

7. A cell according to claim 6, wherein the transcription factor is or comprises Bach2.

8. A cell according to claim 5, wherein the transcription factor comprises a modified version of Bach2 which has reduced or removed capacity to be phosphorylated by ALK.

9. A cell according to claim 8, wherein the transcription factor comprises a modified version of Bach2 with a mutation at one or more of the following positions with reference to the amino acid sequence shown as SEQ ID No. 2: Ser-535, Ser-509, Ser-520.

10. A nucleic acid construct which comprises a first nucleic acid sequence encoding a Chimeric Antigen Receptor (CAR) and a second nucleic acid sequence encoding a transcription factor.

11. A nucleic acid construct according to claim 10, which has the following structure:
CAR-coexpr-TF; or
TF-coexpr-CAR
in which:
CAR is a nucleic acid sequence encoding the CAR;
coexpr is a nucleic acid sequence enabling co-expression of the CAR and the transcription factor; and
TF is a nucleic acid sequence encoding the transcription factor.

12. A nucleic acid construct according to claim 11, wherein coexpr encodes a sequence comprising a self-cleaving peptide.

13. A kit of nucleic acid sequences which comprises a first nucleic acid sequence encoding a Chimeric Antigen Receptor (CAR) and a second nucleic acid sequence encoding a transcription factor.

14. A vector which comprises a nucleic acid construct according to claim 10.

15. A kit of vectors which comprises a first vector which comprises a first nucleic acid sequence encoding a Chimeric Antigen Receptor (CAR); and a second vector which comprises a second nucleic acid sequence encoding a transcription factor.

16. A method for making a cell according to claim 1, which comprises the step of introducing: a nucleic acid construct, a kit of nucleic acid sequences, a vector according to claim 14, or a kit of vectors that comprise(s) a first nucleic acid sequence encoding a Chimeric Antigen Receptor (CAR) and a second nucleic acid sequence encoding a transcription factor, into a cell.

17. (canceled)

18. A pharmaceutical composition comprising a plurality of cells according to claim 1.

19. A method for treating and/or preventing a disease, which comprises the step of administering a pharmaceutical composition according to claim 18 to a subject.

20. A method according to claim 19, which comprises the following steps:
(i) isolation of a cell-containing sample from a subject;
(ii) transduction or transfection of the cells with: a nucleic acid construct, a kit of nucleic acid sequences, a vector, or a kit of vectors that comprise(s) a first nucleic acid sequence encoding a Chimeric Antigen Receptor (CAR) and a second nucleic acid sequence encoding a transcription factor; and
(iii) administering the cells from (ii) to the subject.

21. A method according to claim 19, wherein the disease is a cancer.

22.-23. (canceled)
